(19) United States
(12) Patent Application Publication
(10) Pub. No.: US 2025/0228878 A1
(43) Pub. Date: Jul. 17, 2025

(54) METHOD FOR THE SYNTHESIS OF EVOC PROBES

(71) Applicant: OWLSTONE MEDICAL LIMITED, Cambridge Cambridgeshire (GB)

(72) Inventors: Max Allsworth, Cambridge Cambridgeshire (GB); Christiaan Labuschagne, Cambridge Cambridgeshire (GB)

(21) Appl. No.: 18/700,604

(22) PCT Filed: Oct. 14, 2022
(86) PCT No.: PCT/GB2022/052621

§ 371 Date: Apr. 11, 2024

(30) Foreign Application Priority Data
Oct. 14, 2021 (GB) 2114706.1
Jun. 1, 2022 (GB) 2208155.8

(51) Int. Cl. A61K 31/7028; A61P 35/00; C07H 1/00; C07H 15/18; G01N 33/573; G01N 33/574

(52) U.S. Cl. CPC A61K 31/7028; A61P 35/00; C07H 1/00; C07H 15/18; G01N 33/573; G01N 33/57423; G01N 2333/924; G01N 2474/20

(57) ABSTRACT

A process for the synthesis of a glycoside, comprising the step of;
performing hydrolysis of a methyl ester glycoside in the presence of a base in water, to form a glycoside,
   wherein the propensity to reform the methyl ester glycoside is reduced. The invention also extends to methods for the detection or prognosis of cancer using a glycoside obtained by said process

### INTRODUCTION

Lung cancer is the leading cause of cancer related death world-wide. Early detection of lung cancer is pivotal for patient survival, however due to lack of clinical symptoms in early stages it remains highly challenging and demands for better screening procedures. Despite decades of intense research efforts 5-year survival rates for lung cancer are still dismal at 19% (Siegel et al, 2019). The primary reason for this is the fact that >75% of patients are diagnosed with advanced stage disease where treatment with curative intent is not possible (Walters et al, 2013). At stage I, 1-year survival rates are approximately 80% in contrast to stage IV where it is only 20%. Therefore, early detection of lung cancer is of paramount importance to improve survival rates.

Tumor cells are characterized by metabolic changes during the earliest stages of their development. Measuring the biochemicals related to these metabolic changes can therefore provide diagnostic biomarkers with a potential utility for the early detection of cancer (Muthu & Nordström, 2019). These biomarkers can be detected in biological matrices such as breath, urine and blood. An attractive matrix for detection of these metabolites is breath as it can be accessed fully non-invasively at point of care, therefore lowering the threshold for participation in screening (Hakim et al., 2012).

Recently, the Exogenous Volatile Organic Compound (EVOC) Probe approach has been pioneered which enables active investigation of disease specific pathways. In this approach exogenous metabolic probe compounds are administered to patients which are metabolized by disease specific pathways resulting in a volatile product released in the patient breath (Gaude et al., 2018).

As such there is a need to develop method for the consistent production of EVOC probes that can be used during non-invasive point of care.

### SUMMARY OF THE INVENTION

It has recently been demonstrated that EVOC probes can be used to successfully detect tumors in mice through breath analysis. In this study a metabolic probe, D5-ethyl-βD-glucuronide, which can be metabolized by tumor specific extracellular β-glucuronidase to release D5-ethanol was administered to healthy and tumor bearing mice resulting in a discriminatory D5-ethanol signal in the breath of mice with tumors (Lange et al., 2019). The micro-environment of tumors are known to have high levels of extracellular β-glucuronidase (FISHMAN & ANLYAN, 1947; Young et al., 1976) whilst in normal tissue this enzyme only resides in lysosomes in the cytoplasm (Bosslet et al., 1998). Research in humans and mice has pointed towards a combination of tumor necrosis and release of β-glucuronidase from lysosomes of macrophages and neutrophils in the tumor microenvironment (Bosslet et al., 1998; Juan et al., 2009) as the origins of the extracellular β-glucuronidase in the tumor micro-environment. Therefore, a conjugate of glucuronide with a volatile molecule like D5-ethanol would be cleaved in the tumor micro-environment resulting in the production of glucuronate and D5-ethanol, a readily excretable volatile. Glucuronide-conjugates are not readily imported into the cell when present in the blood at low concentrations. Presence of D5-ethanol in the extracellular space and biological matrices would therefore be considered a hallmark of tumor presence. The inability of D5-ethyl-βD-glucuronide (and other glucuronide conjugates) to diffuse intracellularly at low concentrations is vital for the successful discrimination between healthy and tumor tissue.

However, it is demonstrated herein that the standard process to prepare the glucuronide-based probe results in the production of a methyl ester impurity (D5-ethyl-βD-glucuronide-methyl ester). This methyl ester alters cell permeability and results in uptake of the probe into cells. Within the cell the methyl ester can be cleaved and results in release of D5 ethanol which is not a result of the presence of a tumour. As such this leads to higher background levels of D5 ethanol and potentially to false positive results.

The present inventors have therefore developed a method for the production of the glucuronide, wherein production of the methyl ester impurity is reduced.

As such an aspect of the invention relates to a process for the synthesis of a glycoside, comprising the step of;
performing hydrolysis of a methyl ester glycoside in the presence of a base in water, to form a glycoside,
   wherein the propensity to reform the methyl ester glycoside is reduced.

An aspect of the invention relates to a glycoside obtained by the process of the invention.

An aspect of the invention relates to a composition comprising a glycoside comprising a glycosidic bond linked to a volatile functional group, wherein the composition comprises <1% of a methyl ester glycoside comprising a glycosidic bond linked to a volatile functional group.

An aspect of the invention relates to a kit comprising a glycoside obtained by the process of the invention or a composition comprising a glycoside linked, via a glycosidic bond, to a volatile functional group, wherein the composition comprises <1% of a glycoside methyl ester linked, via a glycosidic bond, to a volatile functional group, and optionally instructions for use.

An aspect of the invention relates to a method of reducing the cell permeability of a glycoside-based exogenous volatile organic compound (EVOC) probe, comprising the step of
applying conditions that promote conversion of a methyl ester glycoside into a glycoside, wherein the conditions that promote formation of the glycoside comprise hydrolysis of the methyl ester glycoside in the presence of a base in water.

An aspect of the invention relates to the use of a glycoside obtained by the process of the invention or a composition comprising a glycoside linked, via a glycosidic bond, to a volatile functional group, wherein the composition comprises <1% of a glycoside methyl ester linked, via a glycosidic bond, to a volatile functional group, in a diagnostic breath test.

In an aspect the invention relates to a method for the detection or prognosis of cancer comprising assessing the activity of a cancer-specific enzyme by measuring the concentration of an exogenous substrate for said enzyme and/or measuring the concentration of a metabolite of said substrate in a biological matrices of a subject, wherein said exogenous substrate is a glycoside obtained according to the invention or a composition according to the invention.

In an aspect the invention relates to a composition comprising a glycoside comprising a glycosidic bond linked to a volatile functional group, wherein the composition comprises <1% of a methyl ester glycoside comprising a glycosidic bond linked to a volatile functional group, for use in a method for the detection or prognosis of cancer comprising assessing the activity of a cancer-specific enzyme by measuring the concentration of an exogenous substrate for said enzyme and/or measuring the concentration of a metabolite of said substrate in a biological matrices of a subject, wherein said exogenous substrate is a composition comprising a glycoside comprising a glycosidic bond linked to a volatile functional group, wherein the composition comprises <1% of a methyl ester glycoside comprising a glycosidic bond linked to a volatile functional group.

In another aspect, the invention relates to a method for the detection or prognosis of cancer comprising assessing the activity of a cancer-specific enzyme by measuring the concentration of an exogenous substrate for said enzyme and/or measuring the concentration of a metabolite of said substrate in a biological matrix of a subject, wherein said exogenous substrate is a glycoside wherein the glycoside is administered at a concentration of 0.05-10 mg/kg and wherein the concentration of the metabolite is measured at up to 300 minutes after administration of the glycoside.

In another aspect, the invention relates to a glycoside for use in the diagnosis or prognosis of cancer comprising administering to a subject a glycoside or composition comprising a glycoside wherein the glycoside is administered at a concentration of 0.05-10 mg/kg.

### FIGURES

The invention is further described in the following non-limiting figures

FIG. 1. Schematic of assay to determine the level of D5 ethanol release in blood samples exposed to D5-ethyl-βD-glucuronide (D5-EG) or D5-ethyl-βD-glucuronide-methyl ester (D5-EG-ME).

FIG. 2. D5-ethanol production from blood cells incubated with either D5-EG or D5-EG-ME

FIG. 3. ¹H NMR of the product formed under standard conditions. The singlet peak at 3.7 is attributed to the methyl ester group

FIG. 4. ¹H NMR of the product formed under conditions which reduce methyl ester content. ¹H NMR (396 MHz, D20, ppm) OH 4.54 (d, J=7.9 Hz, 1H), 4.00 (d, J=9.7 Hz, 1H), 3.59 (t, J=9.1 Hz, 1H), 3.54 (t, J=9.1 Hz, 1H), 3.32 (t, J=8.8 Hz, 1H).

FIG. 5. Schematic of metabolism of D5-ethyl-βD-glucuronide by β-glucuronidase.

FIG. 6. D5 ethanol peak area detected on breath.

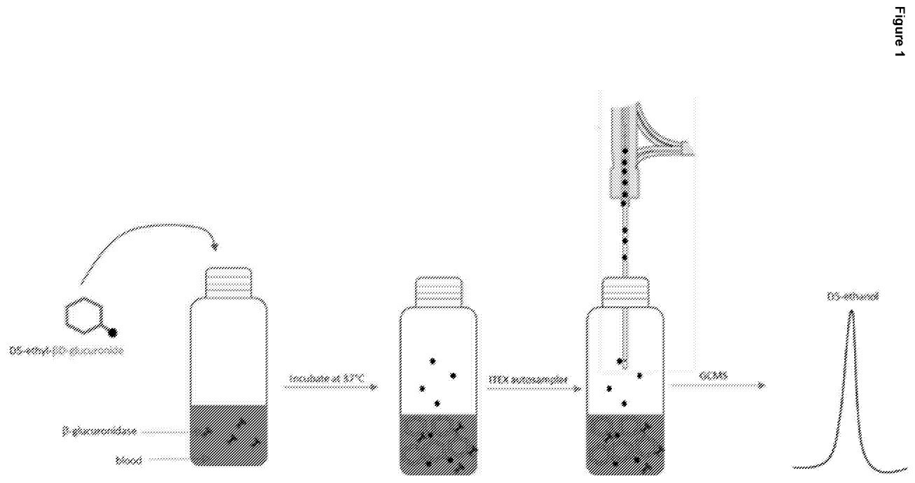

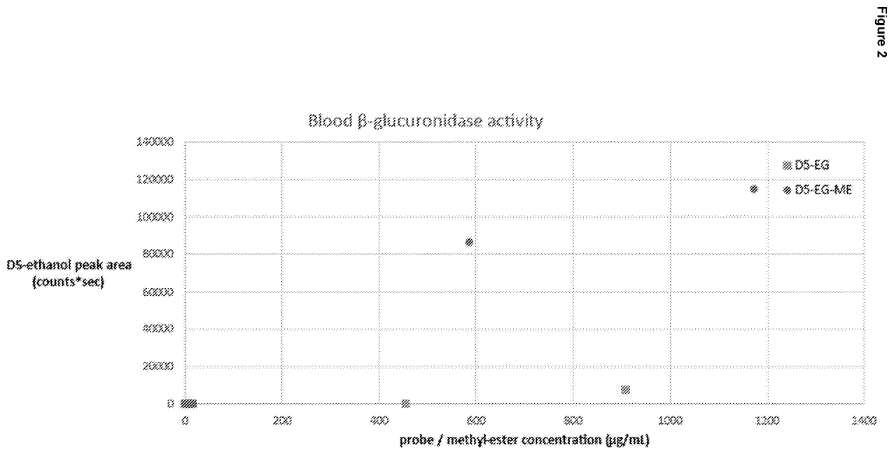

FIG. 7. β-glucuronidase expression in human lung cancer and normal lung tissue.
(A) Representative images of tissue microarrays core tissue microarrays core samples stained for β-glucuronidase expression. β-glucuronidase expression in the tumour microenvironment (TME) was observed at all stages of lung cancer including stage 1. There was no extracellular β-glucuronidase expression in healthy tissue.
(B+C) Quantification of β-glucuronidase expression in lung cancer and normal lung Cancer tissue on tumour microarrays.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The invention relates to a process for the synthesis of a glycoside, comprising the step of;
performing hydrolysis of a methyl ester glycoside in the presence of a base in water, to form a glycoside.

As described herein the present inventors have found that by performing the hydrolysis of the methyl ester glycoside in the presence of a base in water the propensity to reform the methyl ester glycoside is reduced. Glycoside compounds can be used as metabolic probes in the detection of cancer as the glycoside can be metabolized by tumor specific extracellular β-glucuronidase to release a detectable aglycone molecule. However, previous methods to prepare glycoside from methyl ester glycoside have comprised performing hydrolysis in the presence of a base in methanol. It has been shown herein that glycoside produced via this hydrolysis method is able to reform the methyl ester. The presence of the methyl ester glycoside as an impurity increases the cell permeability of the final glycoside. As such the methyl ester glycoside can enter cells and be cleaved by cellular β-glucuronidase. This cleavage is not tumour specific and so could lead to false positives in the detection of cancer. As such by developing a production method wherein the glycoside can no longer reform the methyl ester glycoside the reliability of the glycoside as a metabolic probe is increased.

The hydrolysis of the methyl ester glycoside is suitably performed in the absence of methanol.

The hydrolysis of the methyl ester glycoside is performed in the presence of a base in water. The base may be selected from NaOH, KOH, or LiOH, preferably the base is NaOH. The hydrolysis may be performed at a temperature between 18 to 30° C., or 20 to 29° C., or 22 to 28° C., or 23 to 27° C., or 24 to 26° C. The hydrolysis may be performed at approximately 25° C. Approximately 25° C. may encompass a temperature between 23 to 27° C., or 24 to 26° C., or 24.5 to 25.5° C. The hydrolysis may be performed at a pressure of between 95 to 105 kPa, or 98 to 102 kPa, or 100 to 102 kPa. The hydrolysis may be performed at approximately 101 kPa, wherein approximately 101 kPa may encompass a pressure of between 100 to 102 kPa, or 101 to 102, or 100.5 to 101.5 kPa. The hydrolysis may be performed at approximately 101.325 kPa wherein approximately 101.325 kPa encompasses a pressure between 101.0 to 101.5 kPa. The hydrolysis step may be performed under ambient conditions, wherein ambient conditions comprise a temperature of 25° C. (298.15 K) and pressure of 101.325 kPa.

As used herein the term “glycoside” has its usual meaning in the art and refers to a compound comprising a carbohydrate portion usually a sugar molecule or uronic acid molecule which is linked to a non-sugar molecule via a glycosidic bond. The sugar portion or uronic acid portion may be referred to as the “glycone” and the non-sugar portion may be referred to as the “aglycone”. Glycosides may comprise a six, five or four membered sugar or uronic acid. The glycoside may comprise a monosaccharide, disaccharide, or polysaccharide. In an embodiment the glycoside comprises a carboxylic acid group. Examples of suitable glycosides include but are not limited to glucoside, galactoside, rhamnoside, riboside, arabinoside, fructoside, xyloside, fucoside. Other suitable glycosides include glycosides of uronic acids including glycosides of alluronic acid, altruronic acid, arabinuronic acid, fructuronic acid, glucuronic acid, galacturonic acid, guluronic acid, iduronic acid, lyxuronic acid, mannuronic acid, psicuronic acid, riburonic acid, ribuluronic acid, sorburonic acid, tagaturonic acid, taluronic acid, xyluluronic acid, xyluronic acid. Glycosides of uronic acids may also be referred to as iduronide, mannoside, glucosamide, galactosamide. In a preferred embodiment the glycone is a glycoside of glucuronic acid also referred to as glucuronide.

A glycoside comprises a glycosidic bond linking the glycone (sugar or uronic acid) to an aglycone (non-sugar molecule). The methyl ester glycoside also comprises a glycosidic bond linking the glycone to an aglycone. In an embodiment the aglycone is a volatile compound. As such the glycoside may comprise a glycosidic bond linked to a volatile functional group. The methyl ester glycoside may comprise a glycosidic bond linked to a volatile functional group. Suitable volatile functional groups include but are not limited to ketones, alcohols, carboxylic acids. The functional group when attached to the glycone is not volatile, however once the aglycone is released from the glycone it may become volatile. The aglycone may be released, for example by enzymatic cleavage or acid cleavage.

The term “methyl ester glycoside” refers to a glycoside comprising a methyl ester substituent in place of a hydroxyl or carboxylic acid group on the glycone molecule. In a preferred embodiment, where the glycone molecule is a glucuronide the methyl ester group is attached to the C6 of the glucuronide molecule.

As mentioned above the glycoside comprises a glycosidic bond. The glycosidic bond links the glycone molecule and the aglycone molecule of the glycoside. There are multiple types of glycosidic bond that may be present, for example the glycosidic bond may be selected from an O-, N-, S-, C-glycosidic bond. In an embodiment the glycosidic bond is an O-glycosidic bond. A glycosidic bond is formed between the hemiacetal or hemiketal group of the sugar or uronic acid molecule and the aglycone. The glycosidic bond may adopt an α or a β stereochemistry. The methyl ester glycoside comprises a glycosidic bond.

The volatile functional group may be released from the glycoside under certain conditions, for example in the presence of a certain enzyme or environment conditions such as acidic pH. Where the glycoside is used as a probe in the detection of cancer it is the volatile functional group that is detected. As such in order to improve detection of the volatile functional group the group may be labelled. The volatile functional group may be labelled at a single position in the compound or the volatile functional group may be labelled at multiple positions in the compound. Examples of suitable labels include but are not limited to 12C, 13C, 14C, 2H, 14N or 18O. In an embodiment the volatile functional group is selected from any compound that when cleaved from the glycoside produces a volatile hydroxyl containing group. For example the volatile functional group from methyl, ethyl, propyl, isopropyl, butyl, methyl-D3, ethyl-D5, propyl-D7. When the volatile functional group is cleaved from the glycoside it produces a volatile reporter which can be detected. The volatile reporter is a compound containing a hydroxyl group. The volatile reporter may be selected from methanol, ethanol, propanol, isopropyl alcohol, isobutyl alcohol, butyl alcohol, 2-methyl-3-buten-2-ol, 1-penten-3-ol, isoamyl alcohol, amyl alcohol. The volatile reporter may be labelled as discussed above for example D3-methanol, D5-ethanol, D7-propanol, D7-isopropyl alcohol. The volatile reporter is produced when the volatile functional group is cleaved from the glycoside via cleavage of the glycosidic bond. For example, when the volatile functional group ethyl-D5 is cleaved from the glycoside it produces D5-ethanol.

The process according to the invention may comprise further steps. For example, the process may further comprise a step of ion-exchange. The ion-exchange may be performed after the hydrolysis step is complete. The ion-exchange may be performed using an acidic cation exchange resin, preferably a strong cation exchange resin, suitable resins are known in the art for example Dowex® 50WX8. The resin exchange may be performed in a solvent such as NaOH in water. The process may comprise a step of evaporating the solvent. The step of evaporating the solvent may be performed after the hydrolysis is complete. The step of evaporating the solvent may be performed after the step of resin exchange. In an embodiment the process may further comprise performing both a step of resin exchange and a step of evaporating the solvent. In an embodiment the process comprises;
performing hydrolysis of a methyl ester glycoside in the presence of a base in water, to form a glycoside;
applying the glycoside obtained from the hydrolysis to an ion-exchange resin and performing ion-exchange; and
evaporating the solvent from the glycoside obtained after the ion-exchange, in order to obtain a solid.

In order to determine whether the hydrolysis has gone to completion the reaction can be monitored using various techniques. For example, the hydrolysis reaction may be monitored using a technique such as 1H NMR. By monitoring the hydrolysis of the methyl ester glycoside to form the glycoside, it is possible to determine when the conversion to the glycoside is complete, for example when it is no longer possible to detect the presence of the methyl ester glycoside. As such the process according to the invention may comprise monitoring the hydrolysis of the methyl ester glycoside to form the glycoside, using 1H NMR.

The methyl ester glycoside that is used in the process according to the invention may be obtained by any reasonable route. Where the methyl ester glycoside comprises a glycosidic bond linked to a volatile functional group, the methyl ester glycoside may be obtained by conjugating a methyl ester glycoside to a volatile functional group. For example the methyl ester may be obtained via conjugation of a hexuronic acid with a methyl alcohol, or via reaction of an unsubstituted aldohexuronic acid with methyl alcohol containing dry hydrogen chloride. Suitable methods are outlined in Czalgoszerski, Edward J., “Reactions of Synthesis of Methyl D-Glucuronide” (1934). Master's Theses. 21. In an embodiment, the methyl ester glycoside is obtained by the following steps;
glucuronidation of De ethanol with the bromo sugar 1 using silver carbonate to give the glucopyranoside 2,

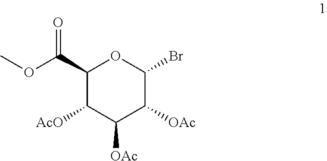 [SMILES: COC(=O)[C@H]1O[C@H](Br)[C@H](OC(C)=O)[C@@H](OC(C)=O)[C@@H]1OC(C)=O]

deacetylating the glucopyranoside 2 using sodium methoxide to give the D5-ethyl-BD-glucuronide-methyl ester 3.

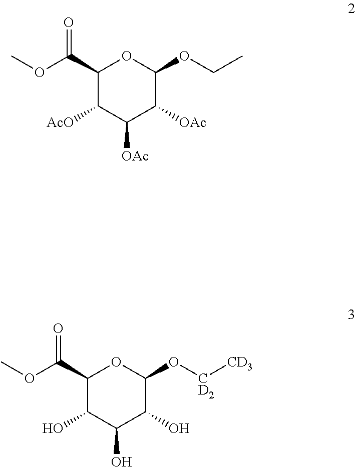

In an embodiment the process for the synthesis of a glycoside, comprises the steps of;
preparing a methyl ester glycoside by any suitable route;
performing hydrolysis of a methyl ester glycoside in the presence of a base in water, to form a glycoside.

According to the invention the hydrolysis of the methyl ester glycoside has been shown to produce a final product comprising the glycoside and <1% of the methyl ester glycoside. In an embodiment after the hydrolysis has been performed the methyl ester glycoside is present at <2%, <1.9%, <1.8%, <1.7%, <1.6%<1.5%, <1.4%, <1.3%, <1.2%, <1.1%, <1%<0.9%, <0.8%, <0.7%, <0.6%, <0.5%, <0.4%, <0.3%, <0.2%, <0.1% of the glycoside. Preferably the methyl ester glycoside is present at <1% of the glycoside. It is possible to determine how much methyl ester glycoside is present in the final product after the hydrolysis has been performed using routine laboratory techniques, for example using 1H NMR. In an embodiment the methyl ester glycoside is present at <1%, <0.9%, <0.8%, <0.7%, <0.6%, <0.5%, <0.4%, <0.3%, <0.2%, or <0.1% of the glycoside as determined by 1H NMR. The % of the methyl ester glycoside may refer to % by weight of the composition. The % of the methyl ester glycoside may refer to % relative to the amount of the glycoside.

In a preferred embodiment the methyl ester glycoside is D5-ethyl-βD-glucuronide-methyl ester. In a preferred embodiment the glycoside is D5-ethyl-βD-glucuronide. As such in a preferred embodiment the invention relates to a process for the synthesis of a D5-ethyl-βD-glucuronide, comprising the step of;
performing hydrolysis of a D5-ethyl-βD-glucuronide-methyl ester in the presence of a base in water, to form a glycoside.

In an aspect the invention relates to a glycoside obtained by the process as defined above. The glycoside obtained from the process of the invention has been shown to have a reduced propensity to reform the methyl ester glycoside intermediate. Preferably the glycoside obtained by the process of the invention comprises a glycosidic bonding linked to a volatile functional group/compound.

In an aspect the invention relates to a composition comprising a glycoside comprising a glycosidic bond linked to a volatile functional group, wherein the composition comprises <2% of a methyl ester glycoside comprising a glycosidic bond linked to a volatile functional group. Preferably the composition comprises <1% of a methyl ester glycoside comprising a glycosidic bond linked to a volatile functional group.

The glycoside present in the composition may have any of the features of the glycoside as defined herein. The methyl ester glycoside present in the composition may have any of the features of the methyl ester glycoside as defined herein.

In an embodiment the composition comprises D5-ethyl-βD-glucuronide, wherein the composition comprises <1% D5-ethyl-βD-glucuronide-methyl ester.

The composition comprises <2%, <1.9%, <1.8%, <1.7%, <1.6%<1.5%, <1.4%, <1.3%, <1.2%, <1.1%, <1%<0.9%, <0.8%, <0.7%, <0.6%, <0.5%, <0.4%, <0.3%, <0.2%, <0.1% of the methyl ester glycoside comprising a glycosidic bond linked to a volatile functional group. The <2%, <1.9%, <1.8%, <1.7%, <1.6%<1.5%, <1.4%, <1.3%, <1.2%, <1.1%, <1%<0.9%, <0.8%, <0.7%, <0.6%, <0.5%, <0.4%, <0.3%, <0.2%, <0.1% of the methyl ester glycoside comprising a glycosidic bond linked to a volatile functional group may be determined using any suitable technique for example it may be determined is via 1H NMR. The % of the methyl ester glycoside may refer to % by weight of the composition. The % of the methyl ester glycoside may refer to % relative to the amount of the glycoside.

The composition of the invention or the glycoside as described herein may be used as a probe in the detection of cancer. As such composition may be suitable for administration via any reasonable route, for example any parenteral or enteral route. For example, any convenient route, includes but is not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation. Parenteral administration includes, for example, intravenous, intramuscular, intraarterial, intraperitoneal, intranasal, rectal, intravesical, intradermal, topical or subcutaneous administration. In an embodiment the composition may be formulated for administration via oral route or inhalation. In an embodiment the composition may be formulated for intravenous administration.

The composition may also include a pharmaceutically acceptable carrier or vehicle. This can be a particulate, so that the compositions are, for example, in tablet or powder form. The term “carrier” refers to a diluent, adjuvant or excipient, with which the glycoside is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. The composition can be in the form of a liquid, e.g., a solution, emulsion or suspension. The liquid can be useful for delivery by injection, infusion (e.g., IV infusion) or sub-cutaneously. As a solid composition for oral administration, the composition can be formulated into a powder, granule, compressed tablet, pill, capsule, chewing gum, wafer or the like form.

The composition may take the form of one or more dosage units. Various oral dosage forms can be used, including such solid forms as tablets, capsules, liquids, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The composition can be in the form of a liquid, e.g., a solution, emulsion or suspension. The liquid compositions, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. The composition can be enclosed in an ampoule, a disposable syringe or a multiple-dose vial made of glass, plastic or other material.

The composition may be formulated for inhalation, as such the composition may be formulated with a suitable particle size and aerodynamic diameter. The glycoside probes of the invention typically have a sufficiently small particle size to enable them to be formulated for inhalation. In other words, the small particle size of the glycoside probes of the invention means that they may be suitable for administration by inhalation. The composition may be optimized for aerosolization. Techniques such as spray-drying and spray-freeze-drying may be used to generate a composition with sufficient powder dispersibility and low agglomeration to be used as an inhaled composition.

The method may be performed by the subject or by a third party, such as by a clinician. In an embodiment, the method may be performed by the subject (or by a third party) in a non-clinical setting. By “non-clinical setting” is meant that a person who is not a trained healthcare professional performs some or all of the steps of the method. For example, one or more of the steps of the method may be performed by the subject at home. For example, the steps of administrating the composition and obtaining the biological sample may be performed by the subject (or by a third party) in a non-clinical setting, such as at home. For example, the biological sample may be sent for external analysis in a clinical setting (such as by a trained healthcare professional or otherwise, as appropriate). In such embodiments, the composition may be formulated for inhalation.

In an embodiment, the method may be performed by a clinician (or other suitable trained healthcare professional). In such embodiments, the composition may be formulated for intravenous administration.

An aspect of the invention relates to a kit comprising a glycoside obtained by the process of the invention or a composition as described herein, and optionally instructions for use.

In an embodiment the kit comprises a device for capturing a biological matrix sample from a subject. The biological matrix can then be analysed for a metabolite of the glycoside obtained by the process of the invention. The device for capturing a biological matrix sample may be a device for capturing a blood sample, a urine sample or an exhaled breath sample. Suitable devices are known in the art.

In one embodiment, the kit further comprises a device for capturing a breath sample from a patient. The device for capturing breath may be as described in WO2017/187120 or WO2017/187141. The device in WO2017/187120 comprises a mask portion which, in use, is positioned over a subject's mouth and nose, so as to capture breath exhaled from the subject. The exhaled breath samples are fed into tubes containing a sorbent material, to which the compounds of interest adsorb. After sufficient sample has been obtained, the sorbent tubes are removed from the sampling device and the adsorbed compounds desorbed (typically by heating) and subjected to analysis to identify the presence and/or amount of any particular compounds or other substances of interest. The preferred analytic technique is field asymmetric ion mobility spectroscopy (abbreviated as “FAIMS”). The method in WO2017/187141 refinement of the method described in WO2017/187120 is disclosed in WO2017/187141. In that document, it is taught to use breath sampling apparatus substantially of the sort described in WO2017/187120, but in a way such as to selectively sample desired portions of a subject's exhaled breath, the rationale being that certain biomarkers or other analytes of interest are relatively enriched in one or more fractions of the exhaled breath, which fractions themselves are relatively enriched in air exhaled from different parts of the subject's body (e.g. nostrils, pharynx, trachea, bronchioles, alveoli etc).

As shown herein the standard conditions used to hydrolyse a methyl ester glycoside into a glycoside has been shown to result in a final product which has the propensity to reform the methyl ester glycoside and as such the product has a higher cell permeability. The standard conditions comprise hydrolysis in the presence of a base in methanol. In contrast the inventors have identified a set of conditions for the hydrolysis of the methyl ester glycoside into a glycoside wherein the glycoside has a reduced propensity to reform the methyl ester glycoside. As such the final product obtained from hydrolysis of the methyl ester glycoside in the presence of a base in water has a reduced cell permeability compared to the glycoside obtained using standard condition.

Therefore, in an aspect the invention relates to a method of reducing the cell permeability of a glycoside-based exogenous volatile organic compound (EVOC) probe, comprising the step of
applying conditions that promote conversion of a methyl ester glycoside into a glycoside, wherein the conditions that promote formation of the glycoside comprise hydrolysis of the methyl ester glycoside in the presence of a base in water.

The term “exogenous volatile organic compound” refers to compounds that, administered to a subject through various routes, undergo metabolism and distribution in the body and are excreted via breath. Additionally, metabolism of EVOCs by cancer-specific enzymes can lead to production of volatile compounds that can also be detected in breath.

In an aspect the invention relates to the use of a glycoside obtained by the process of the invention or a composition as described herein, in a diagnostic breath test. In an embodiment the invention relates to the use of a glycoside obtained by the process of the invention or a composition as described herein, in a breath test that may detect or prognose a disease state in a subject. In an embodiment the invention relates to the glycoside obtained by the process of the invention or a composition as described herein, for use in a breath test that may detect or prognose a disease state in a subject. The disease state may be selected from cancer, preferably lung cancer, or lung inflammation.

The glycoside or the composition comprising the glycoside may be administered at a concentration of 0.05 to 10 mg/kg, for example for 0.5 to 10 mg/kg, for example 0.5-5 mg/kg, for example 1 to 3 mg/kg, for example about 2 mg/kg. In one embodiment, administration is intravenous, by inhalation or oral administration. In one embodiment, administration is intravenous and at 0.5 to 10 mg/kg, for example 0.5-5 mg/kg, for example 1 to 3 mg/kg, for example about 1, 2, 3, 4, 5, 6, 7, 8, 9 or 10 mg/kg. In one embodiment the dosage is 2 mg/kg. In one embodiment, administration is by inhalation or oral administration and at 0.05 to 10 mg/kg, for example 0.5-5 mg/kg, for example 1 to 3 mg/kg, for example about 1, 2, 3, 4, 5, 6, 7, 8, 9 or 10 mg/kg. In one embodiment the dosage is 2 mg/kg.

The breath test may comprise administering the glycoside obtained by the process of the invention or a composition as described herein to a subject, once administered the glycoside is biotransformed by a disease-specific enzyme. The biotransformation of the glycoside may involve the cleavage of the glycosidic bond and therefore the release of the volatile compound. The volatile compound can then be detected in the breath of the subject.

A “subject” as used herein refers to a test subject, e.g. a mammalian subject, preferably a human. In one embodiment, a sample of exhaled breath is obtained for the purpose of diagnosing or screening the presence/absence of a disease. The subject may be a male or female. The subject may be an infant, a toddler, a child, a young adult, an adult or a geriatric. In one embodiment, the subject is a cancer patient, for example lung cancer patient, for example a Stage IIA lung cancer patient.

A disease specific enzyme may be a cancer-specific enzyme. As used herein a “cancer-specific enzyme” is an enzyme that is selected from one or more of the following: the enzyme is absent in cancer tissue, but present in non-cancer tissue; the enzyme is present in cancer tissue, but absent in non-cancer tissue; the enzyme is differentially expressed or in cancer tissue compared to non-cancer tissue or the enzyme is differentially active in cancer tissue compared to non-cancer tissue. For example, the enzyme may be expressed at a higher level in cancer tissue or lower level compared to expression in non-cancer tissue. Expression can be measured by techniques known in the art, for example by mRNA quantification or measuring cDNA. Non-cancer tissue refers for example to healthy tissue. The tissue may be from a specific organ, e.g. lung, colon, breast, prostate etc.

The enzyme may be localised to a different location in cancer tissue compared to non-cancer tissue, for example the enzyme may be present in the extracellular space of cancer tissue, whereas in non-cancer tissue the enzyme is not present in the extracellular space. A combination of tumour necrosis and release of lysosomal enzymes from macrophages and neutrophils can result in the presence of certain enzymes being present in the extracellular space of the tumour microenvironment. In an embodiment the exogenous substrate is a substrate for an enzyme which is present in the extracellular space of solid tumours. The enzyme may be an enzyme which is an extracellular lysosomal enzyme. An “extracellular lysosomal enzyme” is an enzyme that has been released from the lysosome into the extracellular space. Specific example of enzymes that can be released from the lysosome into the extracellular space include-glucuronidase, or β-galactosidase. In particular the presence of β-glucuronidase in the extracellular space is a hall mark of cancer. In an embodiment the cancer-specific enzyme is selected from β-glucuronidase, β-galactosidase α-L-arabinofuranosidase, N-acetyl-β-D-galactosaminidase, N-acetyl-β-D-glucosaminidase, Hexosaminidase, α-L-Fucosidase, α-galactosidase, α-glucosidase, β-glucosidase, α-L-iduronidase, α-mannosidase, β-mannosidase, Lipases, Phosphatases, Sulfatases. In a preferred embodiment the cancer-specific enzyme is selected from β-glucuronidase, β-galactosidase α-L-arabinofuranosidase, N-acetyl-β-D-galactosaminidase, N-acetyl-β-D-glucosaminidase, Hexosaminidase, α-L-Fucosidase, α-galactosidase, α-glucosidase, β-glucosidase, α-L-iduronidase, α-mannosidase, β-mannosidase.

In an embodiment the breath test may be a test for the detection of cancer, optionally lung cancer.

In an aspect the invention relates to a method for the detection or prognosis of cancer comprising assessing the activity of a cancer-specific enzyme by measuring the concentration of an exogenous substrate for said enzyme and/or measuring the concentration of a metabolite of said substrate in a biological matrix of a subject, wherein said exogenous substrate is a glycoside obtained according to the invention or a composition according to the invention.

In an aspect the invention relates to a method for the detection or prognosis of cancer comprising assessing the activity of a cancer-specific enzyme by measuring the concentration of an exogenous substrate for said enzyme and/or measuring the concentration of a metabolite of said substrate in a biological matrix of a subject, wherein said exogenous substrate is a glycoside wherein the glycoside is administered at a concentration of 0.05-10 mg/kg and wherein the concentration of the metabolite is measured at up to 300 minutes after administration of the glycoside.

The glycoside may be administered at a concentration of 0.5-5 mg/kg, for example 1 to 3 mg/kg, about 1, 2, 3, 4, 5, 6, 7, 8, 9 or 10 mg/kg. In one embodiment the dosage is 2 mg/kg. In one embodiment, administration is intravenous, by inhalation or oral administration.

In one embodiment, administration is intravenous and at 0.5 to 10 mg/kg, for example 0.5-5 mg/kg, for example 1 to 3 mg/kg, for example about 2 mg/kg. In one embodiment, administration is by inhalation or oral administration and at 0.05 to 10 mg/kg, for example 0.5-5 mg/kg, for example 1 to 3 mg/kg, for example about 1, 2, 3, 4, 5, 6, 7, 8, 9 or 10 mg/kg. In one embodiment the dosage is 2 mg/kg.

In one embodiment, the concentration of the metabolite is measured at 10 to 30 minutes, 10 to 120, 10 to 180, 30 to 120 or 30 to 180 minutes after administration of the glycoside. In one embodiment, the concentration of the metabolite is measured at 5, 10, 20, 30, 40, 50, 60, 70, 80, 90, 100, 110, 120, 130, 140, 150, 160, 170, 180, 190, 200, 210, 220, 230, 240, 250, 260, 270, 280, 29 or 300 minutes after administration of the glycoside.

In one embodiment, the method comprises the step of correlating the results with beta-glucoronidase expression in a sample, for example as measured by ImmunoHistoChemistry. The sample may be from the subject tested or a subject form a reference sample, e.g. a person that has been diagnosed with cancer or a healthy subject.

The glycoside may be provided as a composition comprising a glycoside comprising a glycosidic bond linked to a volatile functional group, wherein the composition comprises <1% of a methyl ester glycoside comprising a glycosidic bond linked to a volatile functional group as described herein. In one embodiment, the glycoside is obtained by a process for the synthesis of a glycoside, comprising the step of;
performing hydrolysis of a methyl ester glycoside in the presence of a base in water, to form a glycoside,
wherein the propensity to reform the methyl ester glycoside is reduced as described herein.

In an aspect the invention relates to a composition comprising a glycoside comprising a glycosidic bond linked to a volatile functional group, wherein the composition comprises <1% of a methyl ester glycoside comprising a glycosidic bond linked to a volatile functional group, for use in a method for the detection or prognosis of cancer comprising assessing the activity of a cancer-specific enzyme by measuring the concentration of an exogenous substrate for said enzyme and/or measuring the concentration of a metabolite of said substrate in a biological matrices of a subject, wherein said exogenous substrate is a composition comprising a glycoside comprising a glycosidic bond linked to a volatile functional group, wherein the composition comprises <1% of a methyl ester glycoside comprising a glycosidic bond linked to a volatile functional group. In one embodiment, the glycoside may be administered at a concentration of 0.05 to 10 mg/kg, 0.5 to 10 mg/kg, 0.5-5 mg/kg, 1 to 3 mg/kg, 1, 2, 3, 4, 5, 6, 7, 8, 9 or 10 mg/kg. In one embodiment, the dosage is 2 mg/kg.

In one embodiment, administration is intravenous and at, 0.5 to 10 mg/kg, for example 0.5-5 mg/kg, for example 1 to 3 mg/kg, for example about 1, 2, 3, 4, 5, 6, 7, 8, 9 or 10 mg/kg. In one embodiment the dosage is 2 mg/kg. In one embodiment, administration is by inhalation and at 0.05 to 10 mg/kg, for example 0.5-5 mg/kg, for example 1 to 3 mg/kg, for example about 1, 2, 3, 4, 5, 6, 7, 8, 9 or 10 mg/kg. In one embodiment, the dosage is 2 mg/kg. In one embodiment, the concentration of the metabolite is measured at 10 to 30 minutes, 10 to 120 or 10 to 180 minutes after administration of the glycoside.

In one embodiment, the concentration of the metabolite is measured at 10 to 30 minutes, 10 to 120, 10 to 180, 30 to 120 or 30 to 180 minutes after administration of the glycoside. In one embodiment, the concentration of the metabolite is measured at 5, 10, 20, 30, 40, 50, 60, 70, 80, 90, 100, 110, 120, 130, 140, 150, 160, 170, 180, 190, 200, 210, 220, 230, 240, 250, 260, 270, 280, 29 or 300 minutes after administration of the glycoside.

The disease specific enzyme may be an inflammation specific enzyme. In a particular embodiment the enzyme may be specific for lung inflammation. As such the breath test may be used to detect or prognose lung inflammation. In an aspect the invention relates to a method for the detection or prognosis of lung inflammation comprising assessing the activity of a lung inflammation-specific enzyme by measuring the concentration of an exogenous substrate for said enzyme and/or measuring the concentration of a metabolite of said substrate in a biological matrix of a subject, wherein said exogenous substrate is a glycoside obtained according to the invention or a composition according to the invention.

In an aspect the invention relates to a composition comprising a glycoside comprising a glycosidic bond linked to a volatile functional group, wherein the composition comprises <1% of a methyl ester glycoside comprising a glycosidic bond linked to a volatile functional group, for use in a method for the detection or prognosis of lung inflammation comprising assessing the activity of a lung inflammation-specific enzyme by measuring the concentration of an exogenous substrate for said enzyme and/or measuring the concentration of a metabolite of said substrate in a biological matrices of a subject, wherein said exogenous substrate is a composition comprising a glycoside comprising a glycosidic bond linked to a volatile functional group, wherein the composition comprises <1% of a methyl ester glycoside comprising a glycosidic bond linked to a volatile functional group.

The term “lung inflammation” may refer to chronic or acute lung inflammation. Lung inflammation may be caused by an infection such as respiratory infection for example influenza, or respiratory disease such as asthma, bronchitis, chronic obstructive pulmonary disease, or lung cancer.

The term biological matrix refers to a biological sample containing analytes of interest. In an embodiment the biological matrix is selected from blood, urine or exhaled breath. In an embodiment the biological matrix is exhaled breath. Where the biological matrix is blood this may refer to the blood plasma or the blood cells. The sample of biological matrix may be obtained via any reasonable means.

As mentioned above the glycosidic bond is cleavable leading to the release of the volatile functional group from the glycoside. As such the metabolite of the substrate (glycoside or composition of the invention) may be the volatile functional group. The concentration of the substrate and/or metabolite can be measured using methods known in the art. The concentration as used herein means the content or mass of the substrate and/or metabolite in exhaled breath as expressed, for example in grams/litre (g/l). In one embodiment, concentration is measured over time, for example by measuring the kinetics of the clearance. For example, concentration is measured by assessing the kinetic profile of the clearance of the substrate from the biological matrices sample which is then used as a readout. In addition, or alternatively, secretion of metabolic products that can derive from the substrate can be measured over time. For example, clearance of the substrate from biological matrices sample and secretion of metabolic products can both be measured in the same biological matrices sample at the same time or at different times.

The method may comprise the step of providing the exogenous substrate, i.e. the glycoside or composition according to the invention, to the subject. Thus, in one embodiment, the method includes the step of administering the substrate to a subject. Administration may by any convenient route, including but not limited to oral, topical, parenteral, sublingual, rectal, vaginal, ocular, intranasal, pulmonary, intradermal, intravitrial, intramuscular, intraperitoneal, intravenous, subcutaneous, intracerebral, transdermal, transmucosal, by inhalation, or topical, particularly to the ears, nose, eyes, or skin or by inhalation. Parenteral administration includes, for example, intravenous, intramuscular, intraarterial, intraperitoneal, intranasal, rectal, intravesical, intradermal, topical or subcutaneous administration. Preferably, the compositions are administered orally. A skilled person would know that the route of administration depends on the enzyme and disease tested. For instance, if the target enzyme is present in the gastrointestinal tract, oral administration is preferable, while in case of hepatic expression either oral or intravenous administration could constitute viable options.

In one embodiment, the concentration or amount of the substrate and/or its metabolite may be determined in absolute or relative terms in multiple biological matrix samples, for example where the concentration is determined in an exhaled breath sample, it may be determined in a first breath sample (collected at a first time period) and in a second breath sample (collected at a later, second time period), thus permitting analysis of the kinetics or rate of change of concentration thereof over time.

A sample of exhaled breath may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of the substrate (i.e. the glycoside or composition according to the invention) and/or metabolite in a sample of exhaled breath from a subject may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Electronic Nose device, quartz crystal microbalance or chemically sensitive sensors.

The amount of the substrate (i.e. the glycoside or composition according to the invention) and/or metabolite in a sample of exhaled breath from a subject may be determined using thermal desorption-gas chromatography-time of flight-mass spectrometry (GC-Tof-MS). In certain embodiments, breath of the subject is collected in an inert bag, then the content of the bag is transported under standardised conditions onto desorption tubes and VOCs are analyzed by thermally desorbing the content of the tube and then separated by capillary gas chromatography. Then volatile organic peaks are detected with MS and identified using for example a library, such as the National Institute of Standards and Technology. Thermal desorption may be performed at the GC inlet at a temperature of, e.g., about 200-350° C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine one or more VOCs from a sample of exhaled breath from a subject.

In one embodiment, the method comprises collecting different selected exhaled breath samples, or fractions thereof, on a single breath sample capture device, the method comprising the steps of:
(a) collecting a first exhaled breath sample by contacting the sample with a capture device comprising an adsorbent material;
(b) collecting a second exhaled breath sample by contacting the second sample with said capture device, wherein the first and second exhaled breath samples are caused to be captured on the capture device in a spatially separated manner.

In some embodiments, the capture device comprises an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. In one embodiment, the capture device comprises both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

In one embodiment, the methods of the invention further comprise establishing a subject value for one or more substrate and/or metabolite concentration.

In one embodiment, the methods of the invention further comprise comparing the subject value to one or more reference value. In one embodiment, said reference value is from healthy subjects. In another embodiment, the reference value is from subject(s) diagnosed with disease.

In one embodiment, the reference value is a healthy subject value corresponding to values calculated from healthy subjects. In one embodiment, the presence of one or more subject values at quantities greater than their respective range of healthy subject values indicates a substantial likelihood of a disease state in the test subject.

In one embodiment, when an appropriate reference is indicative of a subject being free of a disease, a detectable difference (e.g., a statistically significant difference) between the value determined from a subject in need of characterization or diagnosis of a disease and the appropriate reference may be indicative of the disease in the subject. In one embodiment, when an appropriate reference is indicative of the disease, a lack of a detectable difference (e.g., lack of a statistically significant difference) between the value determined from a subject in need of characterization or diagnosis of a disease and the appropriate reference may be indicative of the disease in the subject.

Thus, in one aspect, the methods include detecting the concentration of the substrate and/or metabolite in a biological matrix sample from the subject, and diagnosing the subject as having a likelihood of a disease state if the level of one or more of the substrate and/or metabolite is different from the healthy subject value.

The methods of the invention may further include the step of selecting a treatment for said disease. The methods may further include administering said treatment to said subject.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

The invention is further described in the following non-limiting examples.

### Example 1-D5-Ethyl-βD-Glucuronide and D5-Ethyl-βD-Glucuronide-Methyl Ester Dose Range in Blood Experimental Protocol

Aim—The following experiment was performed to compare the levels of D5-ethanol produced by blood cells from both D5-ethyl-βD-glucuronide and the D5-ethyl-βD-glucuronide-methyl ester impurity. To understand how the probe behaves in human blood. To determine the background cleavage of the probe (D5-ethyl-βD-glucuronide) and the methyl ester (D5-ethyl-βD-glucuronide-methyl ester) in blood at different concentrations. The cleaved D5-ethanol was extracted from the headspace of the blood and measured with the D5-ethanol analytical method.

### Method-6.1

Dilute D5-ethyl-βD-glucuronide in PBS to 1 ug/ul.
Dilute 10 ul of this in 90 ul PBS to 100 ng/ul to use for smaller doses.
Blood drawn on the same day will be despatched to OML after it has been screened.
Aliquot 600 ul of blood into ITEX headspace vials. 10 vials for probe samples and 10 vials for methyl ester samples.
Add indicated concentration of D5-ethyl-βD-glucuronide (Table 1) and D5-ethyl-bD-glucuronide meth methyl ester (Table 2) in each vial.
Incubate all samples at 37° C. for 2 hours in the water bath.
After 2 hours add 100 uM D-saccharic acid-1,4-lactone with a syringe through septum of each ITEX vial and mix gently to inhibit β-glucuronidase activity and put on ice (4° C.)
Place samples on ITEX autosampler and measure D5-ethanol with D5-ethanol analytical method

TABLE 1
Preparation of probe samples

| | D5-ethyl-βD- | 1 ug/ul | 100 ng/ul |
|---|---|---|---|
| Doses | glucuronide (ug/ml) | stock solution | stock solution |
| Probe 1 | 0.00 | 0 | |
| Probe 2 | 1.33 | — | 8 |
| Probe 3 | 2.67 | — | 16 |
| Probe 4 | 5.33 | 3.2 | |
| Probe 5 | 8.00 | 4.8 | |
| Probe 6 | 10.67 | 6.4 | |
| Probe 7 | 13.33 | 8 | |
| Probe 8 | 26.66 | 16 | |
| Probe 9 | 454.44 | 272.7 | |
| Probe 10 | 908.88 | 545.4 | |

TABLE 2
Preparation of methyl ester samples

| | D5-ethyl-bD- | | |
|---|---|---|---|
| | glucuronide-methyl | 1 ug/ul | 100 ng/ul |
| Doses | ester (ug/ml) | stock solution | stock solution |
| Ester 1 | 0.00 | 0 | |
| Blood 2 | 1.33 | — | 8 |
| Blood 3 | 2.67 | — | 16 |
| Blood 4 | 5.33 | 3.2 | |
| Blood 5 | 8.00 | 4.8 | |
| Blood 6 | 10.67 | 6.4 | |
| Blood 7 | 13.33 | 8 | |
| Blood 8 | 26.66 | 16 | |
| Blood 9 | 586.2 | 351.7 | |
| Blood 10 | 1172.4 | 703.4 | |

Results—β-glucuronidase is only expected to be present intracellularly in blood cells. D5-ethyl-βD-glucuronide (D5-EG) is not cell permeable and would therefore not enter into the cells to get cleaved by b-glucuronidase to produce D5-ethanol. Low D5-ethanol levels are therefore expected in blood when incubated with D5-ethyl-βD-glucuronide.

D5-ethyl-βD-glucuronide-methyl ester (D5-EG-ME) is expected to be more cell permeable. If this is the case it would be able to enter into the cell to get cleaved by intracellular β-glucuronidase resulting in an increased in D5-ethanol production.

Indeed, when blood cells are incubated with D5-EG only a small amount of D5-ethanol were produced in the highest concentration tested (FIG. 2). In contrast, when blood cells were incubated with D5-EG-ME there was much higher D5-ethanol levels produced in the highest concentration and the second highest concentration tested (FIG. 2).

The presence of the methyl ester would therefore result in D5-ethanol produced by normal cells which is unfavourable for a probe to detect lung cancer.

### Example 2—Identification of Conditions that Reduce Methyl Ester Content

The conditions to convert the D5-EG-ME into D5-EG comprise hydrolysis of D5-EG-ME in the presence of base in methanol as set out below:

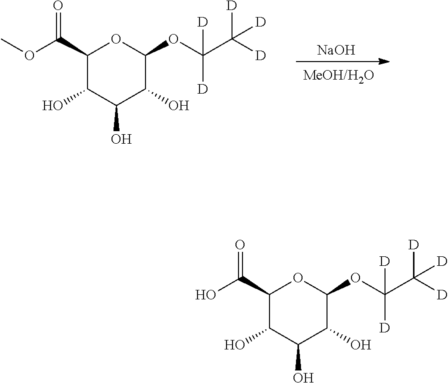

The reaction is monitored via proton NMR to ensure no residual D5-EG-ME remains in the product (D5-EG). The D5-EG was isolated as a white solid after a cation exchange and a series of water and methanol co-evaporations. Under these process conditions it was found that the reverse reaction is possible, and the methyl ester can be reformed. As such presence of the methyl-ester as an impurity in the D5-EG when used as a probe leads to higher levels of D5 ethanol release which is due to the higher cell permeability of the D5-EG-ME. This would lead to higher background level of D5 ethanol when this product is used as an EVOC probe sin the detection of lung cancer and could lead to false positives.

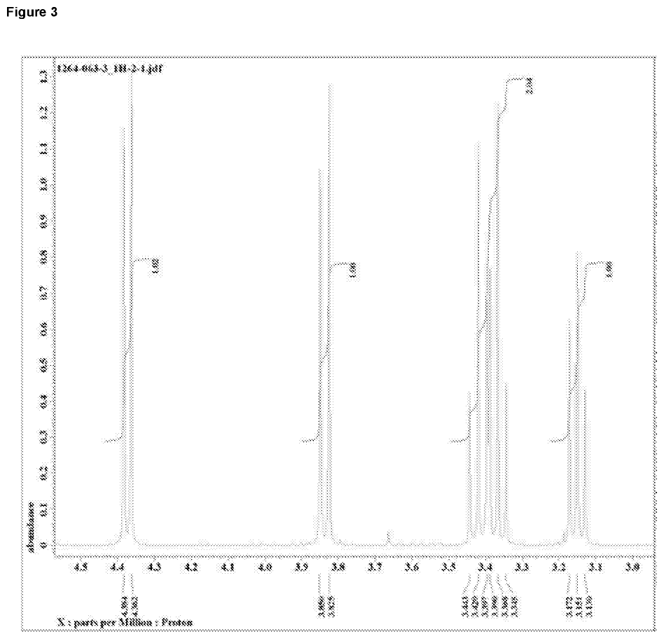

¹HNMR was used to characterise the product after hydrolysis, resin exchange and water and methanol co-evaporations. It was shown that the D5-EG-ME was present at levels of 1% within the final product. The NMR spectrum in FIG. 3 shows a singlet peak at 3.7 which is indicative of the methyl ester.

As such identification of conditions to reduce the reformation of the D5-EG-ME, was carried out. It was surprisingly found that by performing the hydrolysis of the D5-EG-ME in the presence of base in water under ambient conditions as shown below:

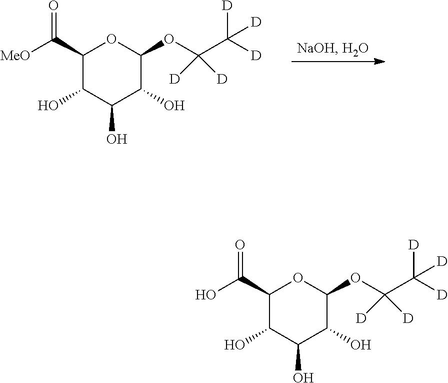

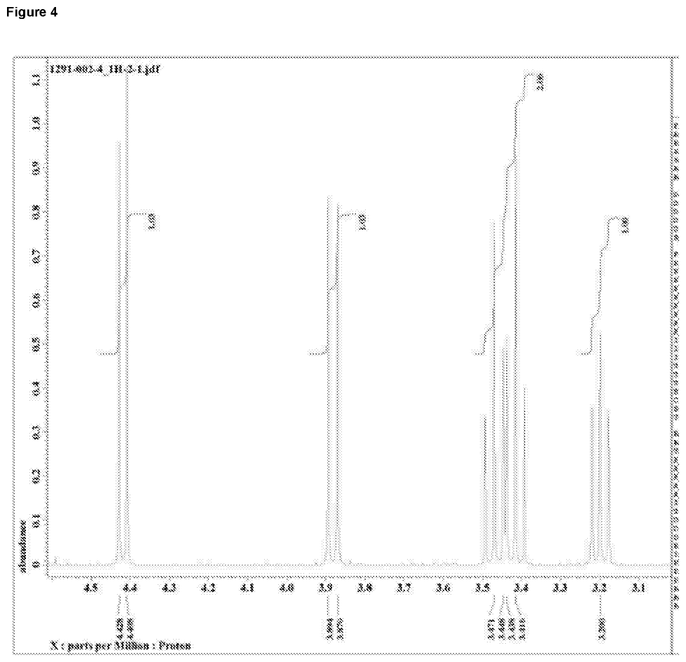

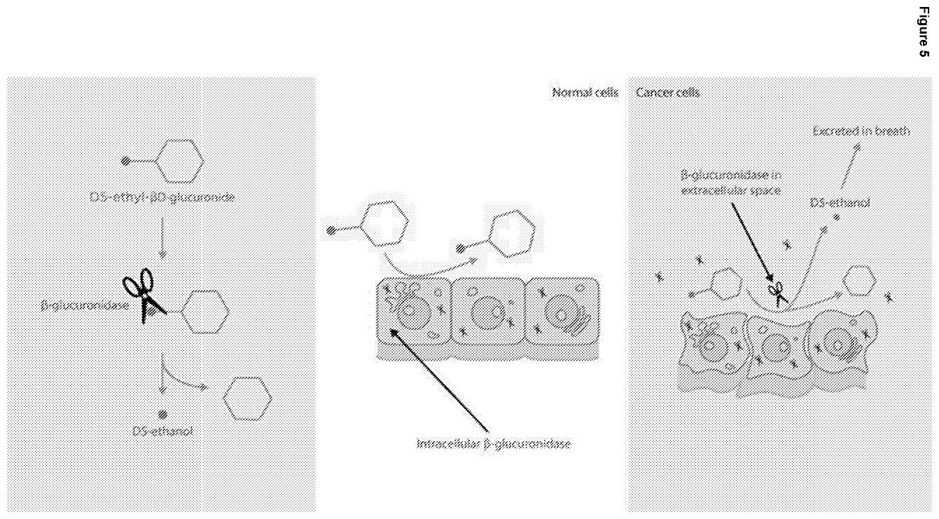

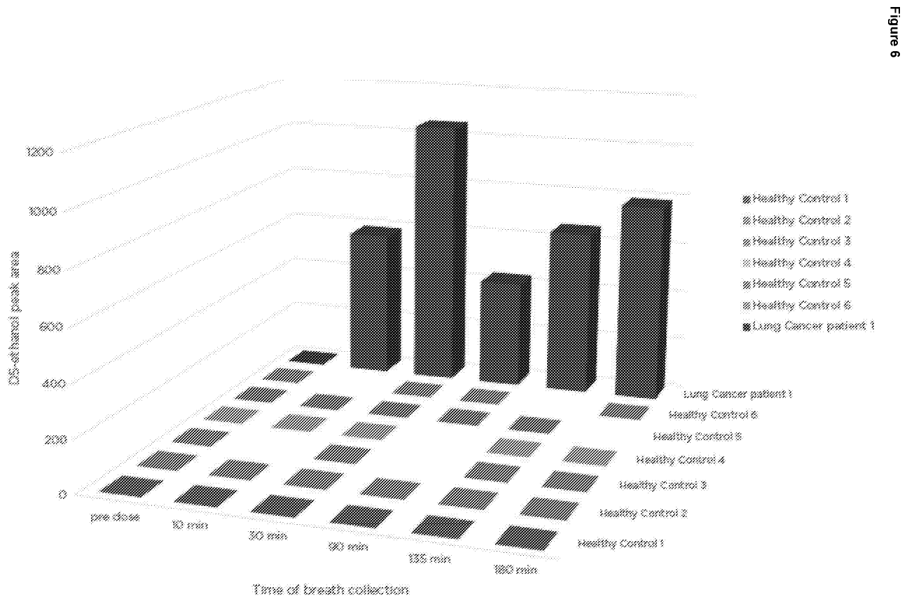

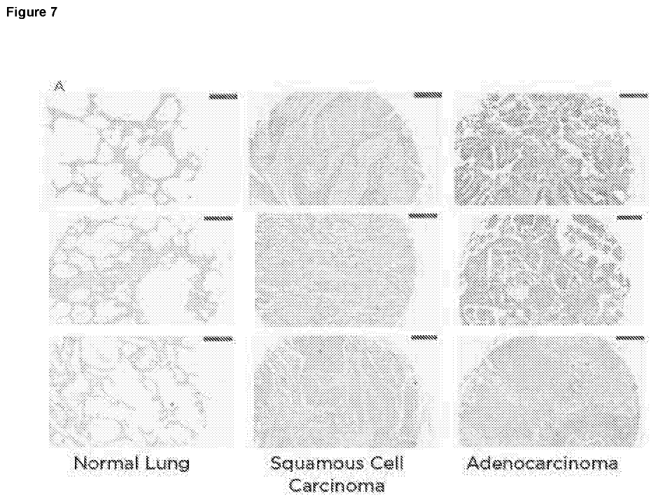

The reaction was monitored via proton NMR to ensure no residual INT_005_0012 remains in the product. The product is isolated as a white solid after a resin exchange and evaporation of the water solvent. Under these process conditions methanol is not present and the methyl ester is not reformed during product isolation. This enables the methyl ester impurity to be controlled to levels of less than 1% by proton NMR. The ¹H MNR spectrum in FIG. 4 shows that there is no singlet peak at 3.7 indicative of the methyl ester.

### Example 3—Large Scale Hydrolysis

### Procedure

D5-EG-ME was suspended in water (22 mL) and stirred at r.t. for 5 min forming a clear light yellow solution. NaOH in water (22 mL) was then added dropwise to form a darker yellow solution. The reaction mixture was stirred for ˜3 h.

The reaction mixture was analysed 1H NMR.

1H NMR: 50 μL of sample taken and added to water (100 uL) with approx. 25 mg Dowex. Left to sit for 15 min and sample concentrated.

No evidence of D5-EG-ME by absence of methyl peak that should be at approx. 3.73 ppm. To reaction mixture was added pre-washed Dowex® 50WX8 50-100 (H) (18 g).

Stirred at r.t. for 2.5 h and filtered. pH of filtrate: 3.8, yellow solution. Dowex washed with water (2×30 mL). To filtrate was added pre-washed Dowex® 50WX8 50-100 (H) (18 g). Stirred at r.t. for 90 min and filtered. pH of filtrate: 2.2 (pH on meter still dropping) lighter yellow solution. Dowex washed with water (2×30 mL).

Filtrate concentrated to dryness (up to 55° C. and up to 2 h) to give a cream solid. Yield=7.94 g (95%)

### Example 4—Preparation of D5-Ethyl-βD-Glucuronide-Methyl Ester

The starting material D5-ethyl-βD-glucuronide-methyl ester was prepared according to the following method.

Step 1—Glucuronidation of De Ethanol with the bromo sugar 1 using silver carbonate to give the glucopyranoside 2.

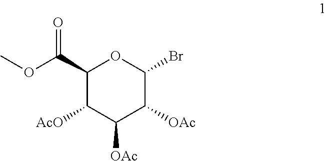 [SMILES: COC(=O)[C@H]1O[C@H](Br)[C@H](OC(C)=O)[C@@H](OC(C)=O)[C@@H]1OC(C)=O]

Step 2—Deacetylation of the glucopyranoside 2 using sodium methoxide to give the D5-ethyl-βD-glucuronide-methyl ester 3.

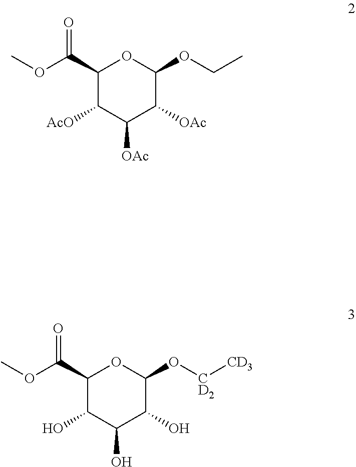

Once the methyl ester is prepared the hydrolysis of the methyl ester can be performed according to the present invention.

### Example 5 Administration of Probe

D5-ethyl-βD-glucuronide was administered to healthy subjects and a lung cancer patient at a concentration of 2 mg/kg. Administration was intravenous.

The concentration of the metabolite was then measured in breath samples. Breath samples were collected using ReCIVA® Breath Sampler at the indicated times following probe administration. Following breath collection sorbent tubes were removed from ReCIVA®, capped off and sent to Owlstone Medical for analysis. At Owlstone Medical the sorbent tubes were analysed for D5-ethanol using TD-GC-MS.

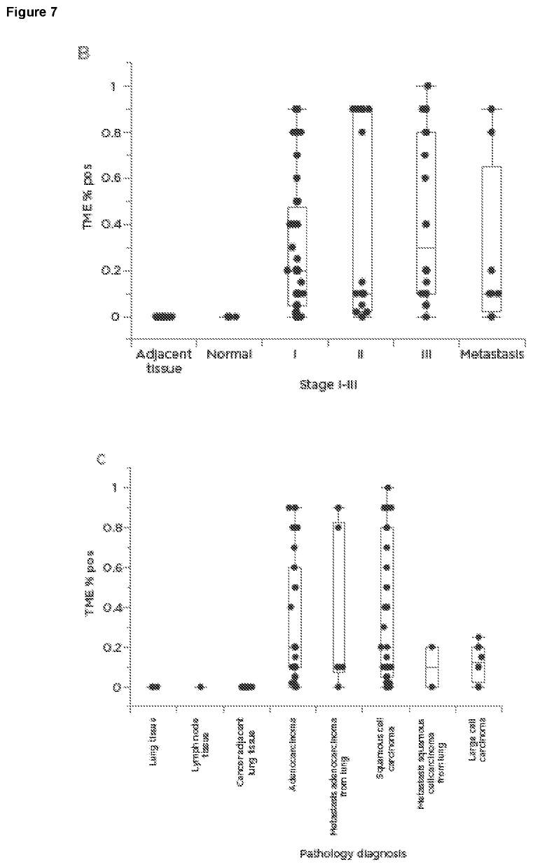

No D5-ethanol was detected in healthy controls following probe administration. A D5 ethanol signal was detected in the lung cancer patient (Stage IIA). As shown in FIG. 6, the metabolite can be detected as early as after 10 minutes from administration of the probe.

### Example 6

β-glucuronidase expression was measured in human lung cancer and normal lung tissue using ImmunoHistoChemistry. The results are shown in FIG. 7.

### REFERENCES

Bosslet, K., Strä, R., Blumrich, M., Czech, J., Gerken, M., Sperker, B., Kroemer, H. K., Gesson, J., Koch, M., & Monneret, C. (1998). Elucidation of the Mechanism Enabling Tumor Selective Prodrug Monotherapyl. 14, 1195-1202.
FISHMAN, W. H., & ANLYAN, A. J. (1947). The presence of high beta-glucuronidase activity in cancer tissue. The Journal of Biological Chemistry, 169 (2), 449.
Gaude, E., Patassini, S., Boschmans, J., Nakhleh, M. K., & van der Schee, M. P. (2018). Targeted breath analysis: exogenous volatile organic compounds (EVOC) as targeted metabolic probes in Breath Biopsy. Journal of Breath Research, 13 (3), 032001.
Hakim, M., Broza, Y. Y., Barash, O., Peled, N., Phillips, M., Amann, A., & Haick, H. (2012). Volatile organic compounds of lung cancer and possible biochemical pathways. In Chemical Reviews. https://doi.org/10.1021/cr300174a
Juan, T., Roffler, S. R., Hou, H., Huang, S., Chen, K., Leu, Y., Prijovich, Z. M., & Wu, C. (2009). Antiangiogenesis Targeting Tumor Microenvironment Synergizes Glucuronide Prodrug Antitumor Activity. 9 (10), 4600-4612. https://doi.org/10.1158/1078-0432.CCR-09-0090
Lange, J., Eddhif, B., Tarighi, M., Garandeau, T., Péraudeau, E., Clarhaut, J., Renoux, B., Papot, S., & Poinot, P. (2019). Volatile Organic Compound Based Probe for Induced Volatolomics of Cancers. Angewandte Chemie (International Ed. in English), 58 (49), 17563-17566. https://doi.org/10.1002/anie.201906261
Muthu, M., & Nordstrom, A. (2019). Current status and future prospects of clinically exploiting cancer-specific metabolism—why is tumor metabolism not more extensively translated into clinical targets and biomarkers? In International Journal of Molecular Sciences. https://doi.org/10.3390/ijms20061385
Young, C. W., Yagoda, A., Bittar, E. S., Smith, S. W., Grabstald, H., & Whitmore, W. (1976). Therapeutic trial of aniline mustard in patients with advanced cancer. Comparison of therapeutic response with cytochemical assessment of tumor cell beta-glucuronidase activity. Cancer, 38 (5), 1887-1895. https://doi.org/10.1002/1097-0142 (197611) 38:5<1887::aid-cncr2820380504>3.0.co;2-#

1. A process for the synthesis of a glycoside, comprising the step of;
performing hydrolysis of a methyl ester glycoside in the presence of a base in water, to form a glycoside,
wherein the propensity to reform the methyl ester glycoside is reduced.

2. The process according to claim 1 wherein the base is NaOH, LiOH, KOH.

3. The process according to claim 1 or 2, wherein the glycoside is selected from glucuronide, iduronide, mannoside, glucosamide, galactosamide, glucoside, galactoside, rhamnoside, riboside, arabinoside, fructoside, xyloside, fucoside.

4. The process according to any one of claims 1-3, wherein the glycoside comprises a glycosidic bond linked to a volatile functional group.

5. The process according to claim 4, wherein the glycosidic bond is selected from an O-, N-, S-, C-glycosidic bond.

6. The process according to claim 4 or 5, wherein the volatile functional group is labelled.

7. The process according to claim 5, wherein the label is selected from 12C, 13C, 14C, 2H, 14N or 18O.

8. The process according to any one of claims 4-7, wherein the volatile functional group is selected from methyl, ethyl, propyl, isopropyl, butyl, methyl-D3, ethyl-D5, or propyl-D7.

9. The process according to any one of claims 1-8, further comprising a step of ion-exchange after the step of hydrolysis, preferably wherein the ion-exchange is performed using an acidic cation exchange resin.

10. The process according to any one of claims 1-9, further comprising a step of evaporating the solvent, after the step of hydrolysis.

11. The process according to any one of claims 1-10, wherein the method comprises monitoring the hydrolysis of the methyl ester glycoside to form the glycoside, using ¹H NMR.

12. The process according to any one of claims 1-11, wherein after the step of hydrolysis, the methyl ester glycoside is present at <1% of the glycoside.

13. The process of claim any one of claims 1-12, wherein the hydrolysis is performed at a temperature between 18 to 30° C.

14. The process according to any one of claims 1-13 wherein the hydrolysis is performed under ambient conditions comprising a temperature of 25° C. (298.15 K) and pressure of 101.325 kPa.

15. The process according to any one of claims 1-14 wherein the methyl ester glycoside is D5-ethyl-βD-glucuronide-methyl ester, and wherein the glycoside is D5-ethyl-BD-glucuronide.

16. A glycoside obtained by the process of any one of claims 1-15.

17. A composition comprising a glycoside according to claim 16.

18. A composition comprising a glycoside comprising a glycosidic bond linked to a volatile functional group, wherein the composition comprises <1 wt % of a methyl ester glycoside comprising a glycosidic bond linked to a volatile functional group.

19. A composition according to claim 17 or 18, wherein the <1 wt % of a methyl ester glycoside comprising a glycosidic bond linked to a volatile functional group, is determined via ¹H NMR.

20. A glycoside according to claim 16 for use in the diagnosis or prognosis of cancer comprising administering to a subject the glycoside according to claim 16 or the composition according to any one of claims 17-19.

21. A glycoside for use according to claim 20, wherein the glycoside is administered at a concentration of 0.05 to 10 mg/kg.

22. A glycoside for use according to claim 20 or 21, wherein administration is intravenous and the concentration is 0.5 to 10 mg/kg or wherein administration is by inhalation or oral administration and the concentration is 0.05 to 10 mg/kg.

23. A glycoside for use according to any of claims 20-22, wherein the concentration of a metabolite of the glycoside is measured up to 300 minutes after administration of the glycoside.

24. A method of reducing the cell permeability of a glycoside-based exogenous volatile organic compound (EVOC) probe, comprising the step of
applying conditions that promote conversion of a methyl ester glycoside into a glycoside,
wherein the conditions that promote formation of the glycoside comprise hydrolysis of the methyl ester glycoside in the presence of a base in water.

25. Use of a glycoside according to claim 16 or a composition according to any of claims 17-19 in a breath test for the detection or prognosis of a disease state.

26. Use according to claim 25, wherein the breath test is for the detection of cancer, optionally lung cancer.

27. A method for the detection or prognosis of cancer comprising assessing the activity of a cancer-specific enzyme by measuring the concentration of an exogenous substrate for said enzyme and/or measuring the concentration of a metabolite of said substrate in a biological matrix of a subject, wherein said exogenous substrate is a glycoside according to claim 16.

28. A method according to claim 27, comprising the step of administrating a glycoside according to claim 16 or a composition according to any of claims 17-19 to the subject.

29. A method for determining the activity of an enzyme comprising;
administering an exogenous substrate for an enzyme to a subject,
measuring the concentration of a metabolite of said substrate in a biological sample that has been obtained from said subject;
wherein said exogenous substrate is a glycoside according to claim 16.

30. A method for monitoring the progression of cancer in a subject diagnosed with cancer comprising:
administering an exogenous substrate to a subject,
assessing the activity of a cancer-specific enzyme by measuring the concentration of a metabolite of said substrate in a biological sample obtained from the subject;
wherein said exogenous substrate is a glycoside according to claim 16.

31. A method for determining efficacy of a treatment comprising in a subject diagnosed with cancer:
administering an exogenous substrate to a subject,
assessing the activity of a cancer-specific enzyme by measuring the concentration of a metabolite of said substrate in a biological sample obtained from the subject wherein said subject has received anti-cancer treatment;
wherein said exogenous substrate is a glycoside according to claim 16.

32. The method according to any one of claims 27-31, wherein the glycoside is D5-ethyl-BD-glucuronide.

33. The method according to claim 32, wherein the metabolite is D5 ethanol.

34. The method according to any one of claims 27-33, wherein the cancer-specific enzyme is β-glucuronidase,

35. The method according to any one of claims 27-34, wherein the biological matrix is selected from blood, urine or exhaled breath, for example exhaled breath.

36. The method according to any one of claims 27-35, wherein the glycoside is administered at a concentration of 0.05-10 mg/kg.

37. The method according to any one of claims 27-36, wherein the concentration of the metabolite is measured at up to 300 minutes after administration of the glycoside.

38. The method according to any one of claims 27-37, wherein administration is intravenous administration and the concentration is 0.5 to 10 mg/kg or by inhalation or oral administration and the concentration is 0.05 to 10 mg/kg.

39. The method according to any one of claims 27-38, wherein the concentration is 1 mg/kg to 5 mg/kg, for example about 2 mg/kg.

40. The method according to any one of claims 27-39, wherein the concentration of the metabolite is measured at 10 to 30 minutes after administration of the glycoside.

41. A glycoside for use according to any of claims 21-23 or a method according to any of claims 27-40, comprising the step of correlating the results with beta-glucoronidase expression, for example as measured by ImmunoHistoChemistry (IHC).

42. A kit comprising a glycoside according to claim 16 or a composition according to any of claims 17-19, and optionally instructions for use.

43. A kit according to claim 42, further comprising a device for capturing a biological matrix sample from a patient, preferably wherein the device is suitable for capturing a sample of blood, urine or exhaled breath.
